(19) United States
(12) Patent Application Publication
(10) Pub. No.: US 2013/0289089 A1
(43) Pub. Date: Oct. 31, 2013

(54) Pharmaceutical Compositions for Topical Delivery of Photosensitizers and Uses Thereof

(71) Applicants: Jerome A. Morris, Berthoud, CO (US); Deepank Utkhede, Surrey (CA); David W.C. Hunt, Surrey (CA)

(72) Inventors: Jerome A. Morris, Berthoud, CO (US); Deepank Utkhede, Surrey (CA); David W.C. Hunt, Surrey (CA)

(21) Appl. No.: 13/979,315

(22) PCT Filed: Jan. 13, 2012
(86) PCT No.: PCT/US12/21263

§ 371 Date: Jul. 11, 2013

Related U.S. Application Data
(60) Provisional application No. 61/432,453, filed on Jan. 13, 2011.

(51) Int. Cl. A61K 41/00; A61K 9/00

(52) U.S. Cl. CPC A61K 41/0071; A61K 9/0014
USPC 514/410

(57) ABSTRACT

The invention includes and provides compositions comprising photo [symbol] sensitizing agents, in particular lemuteporfin, and their use in photo-dynamic therapy for the treatment of dermatological conditions.

### FIELD OF THE INVENTION

The present invention includes and provides compositions comprising photosensitizing agents and their use in photodynamic therapy for the treatment of dermatological conditions.

### BACKGROUND OF THE INVENTION

Photodynamic therapy (PDT) is a procedure that uses light-activated drugs (photosensitizers) to treat a wide range of medical conditions. Accumulation of the photosensitizer in a target tissue that can be directly illuminated makes PDT a selective treatment. When a photosensitizer is activated by light, singlet oxygen and other free radicals are produced in tissues that have retained the drug. The interaction of these reactive oxygen species with biological macromolecules induces a cascade of biochemical reactions that cause changes in cell metabolism, and at high doses of drug and/or light, can result in cell death.

Photodynamic therapy (PDT) has been proposed as a treatment for a number of skin conditions, including acne vulgaris, hyperactive sebaceous glands, psoriasis, atopic dermatitis, and certain types of skin cancers. One of the challenges in performing PDT for these conditions has been targeting sufficient quantities of photosensitizer to the desired location in the skin without causing generalized and unwanted skin photosensitivity reactions such as erythema, pain, burning and itching after irradiation with light. For example, in treating conditions such as acne vulgaris, sebaceous gland hyperplasia, seborrhea and seborrheic dermatitis, conditions characterized by sebaceous gland hyperactivity, it would be desirable to have the photosensitizer drug selectively localized in the sebaceous glands.

A number of topical formulations of photosensitizers have been proposed for treating skin conditions (see for example, WO 2005/074987). Formulation composition may markedly influence topical photosensitizer delivery into the skin and potentially skin appendages such as pilosebaceous units (PSU), structures consisting of a hair follicle with associated sebaceous glands. There is a need for better formulations that effectively deliver photosensitizer drugs into sebaceous glands.

### SUMMARY OF THE INVENTION

In one aspect, the present invention provides pharmaceutical compositions comprising photosensitizers, and methods of using the compositions to perform photodynamic therapy (PDT) for the treatment of dermatological disorders such as acne vulgaris and other hyperactive sebaceous gland disorders.

The invention also includes and provides a pharmaceutical composition comprising, in a solution, a photosensitizer and one or more pharmaceutically acceptable excipients, wherein the solution is supersaturated with the photosensitizer and wherein the photosensitizer does not precipitate out of solution to a pharmaceutically unacceptable degree prior to use.

The invention also includes and provides a pharmaceutical composition comprising, in a solution, a photosensitizer and one or more pharmaceutically acceptable excipients, wherein the solution is supersaturated with the photosensitizer and wherein the photosensitizer does not precipitate out of solution for a period of at least four hours after the solution is made.

In another aspect the invention includes and provides a pharmaceutical composition useful for localizing a photosensitizer to a sebaceous gland, comprising (1) a photosensitizing component comprising a photosensitizer, and (2) an excipient component, together in a solution, wherein the concentration of the photosensitizer in the solution is supersaturating.

In another aspect the invention includes and provides a pharmaceutical composition useful for localizing a photosensitizer to a sebaceous gland, comprising (1) a photosensitizing component comprising a photosensitizer, and (2) an excipient component, together in a solution, wherein the concentration of the photosensitizer in the solution is supersaturating, and wherein the photosensitizer does not precipitate out of solution to a pharmaceutically unacceptable degree.

The invention further includes and provides a pharmaceutical composition comprising a solubilized photosensitizer and one or more excipients, wherein the concentration of photosensitizer in the composition exceeds the solubility of the photosensitizer in the solution.

The present invention further includes and provides a composition useful for delivery of a photosensitizer to a sebaceous gland, comprising a photosensitizer, one or more solvents and optionally one or more pharmaceutically acceptable excipients, wherein the composition has a viscosity of less than 50 centipoise (cps) at 20° C.

The invention also provides a topical formulation effective for localizing a photosensitizer to a sebaceous gland comprising: (1) a photosensitizing component comprising a photosensitizer; and associated therewith but separate therefrom, (2) an excipient component,

wherein the photosensitizer is present in an amount sufficient to form, on mixing, a supersaturated solution thereof, and wherein the photosensitizer does not precipitate out of solution to a pharmaceutically unacceptable degree once components (1) and (2) are mixed.

The invention further includes and provides a two-component pharmaceutical composition comprising two liquid phases, wherein at least one of the liquid phases comprises a photosensitizer dissolved therein, the two liquid phases are miscible, and the photosensitizer has different solubilities in the first liquid phase and the second liquid phase, and wherein the concentration of the photosensitizer in each liquid phase is such that, upon combination of the two liquid phases, the total photosensitizer concentration in the liquid mixture is greater than the solubility of the photosensitizer in the liquid mixture, whereby the resulting liquid mixture is supersaturated with the photosensitizer.

The invention further includes and provides a two-component pharmaceutical composition comprising a first liquid phase and a second liquid phase that are initially physically separated but are intended to be combined together to form a liquid mixture prior to use, wherein: at least one of the liquid phases comprises a photosensitizer dissolved therein, the two liquid phases are different but are miscible, and the solubility limit of the photosensitizer in the first liquid phase and the second liquid phase are different; and wherein the concentration of the photosensitizer in each liquid phase is such that, initially upon mixing of the two liquid phases, the total photosensitizer concentration in the liquid mixture is greater than the saturation concentration of the photosensitizer in the liquid mixture, whereby the resulting liquid mixture is supersaturated with the photosensitizer.

The invention also includes and provides methods for reducing the sebum excretion rate by sebaceous glands of a subject in need thereof, comprising topically applying a therapeutically effective amount of a photosensitizer composition of the invention to the skin of the subject, allowing sufficient time for at least some of the photosensitizer to localize in the sebaceous glands, and exposing the skin of the subject to light energy at a wavelength capable of activating the photosensitizer.

The invention also includes and provides methods for treating a hyperactive sebaceous gland disorder in an affected area of the skin of a subject in need thereof, comprising topically applying a therapeutically effective amount of a photosensitizer composition of the invention to the affected area of the skin of the subject, allowing sufficient time for at least some of the photosensitizer to localize in the sebaceous glands, and exposing the skin of the subject to light energy at a wavelength capable of activating the photosensitizer. Preferred hyperactive sebaceous gland disorders include acne (including acne vulgaris), seborrhea (or oily skin), seborrheic dermatitis, hidradenitis suppurativa (acne inversa), and sebaceous gland hyperplasia.

The invention also includes and provides methods of treating acne in a subject in need thereof, comprising topically applying a therapeutically effective amount of a photosensitizer composition of the invention, allowing sufficient time for at least some of the photosensitizer to localize in the sebaceous glands of the subject, and exposing the skin of the subject to light energy at a wavelength capable of activating the photosensitizer.

The invention also includes and provides methods for ablating sebocytes in a subject afflicted with a hyperactive sebaceous gland disorder such as acne, comprising the steps of delivering a therapeutically effective amount of a photosensitizer to the sebocytes of the subject, allowing sufficient time for the photosensitizer to localize in the sebocytes, and exposing the sebocytes to light energy at a wavelength capable of activating the photosensitizer.

The invention also includes and provides a kit comprising a first container containing a photosensitizing component comprising a photosensitizer, and a second container containing an excipient component that is miscible with the solvents in the first container, and a set of instructions for combining the contents of the two containers, topically applying the combined contents to the skin of a subject, and performing PDT for the treatment of one or more skin disorders.

Preferred photosensitizers include green porphyrins such as lemuteporfin and verteporfin.

### BRIEF DESCRIPTION OF THE DRAWINGS

FIG. 1 is a graph showing the effect on mouse sebaceous glands of PDT with various solution formulations of lemuteporfin (LT-G-001-LT-G-005 shown in Table 3; with and without cellulose gelling agents) and an ointment formulation (LTO-TG1) with red light doses of 50 or 100 J/cm² delivered at an intensity of 50 mW/cm². Flank skin samples obtained 72 hours post-PDT were assessed for numbers of Oil Red O-positive PSU (□) which indicates the presence of sebaceous glands, and the total number of hair follicles (▪) counted within each 4× microscopic field. Mean values with standard deviations for 5 mice per treatment group are presented.

FIG. 2 is a graph comparing the effect of PDT with lemuteporfin in a lemuteporfin topical solution (LTS; LT-G-002-type) in comparison to a lemuteporfin topical ointment (LTO; TG1-type) combined with red light doses of 20, 50 or 100 J/cm² at an intensity of 50 mW/cm². Control mice received an application of matched formulation that did not contain lemuteporfin and then were exposed to the highest red light dose. Sections prepared from flank skin samples were obtained 72 hours post-PDT were assessed for Oil Red O-positive PSU (□) and total hair follicles (▪) within each 4× microscopic field. Mean values with standard deviations for 5 mice per treatment group are presented.

FIG. 3 is a bar graph showing lemuteporfin fluorescence intensity measurement in hair follicles and sebaceous glands in human cadaver skin samples comparing a lemuteporfin topical ointment (LTO) at 1 hour and 8 hours after application of lemuteporfin-containing formulation and a lemuteporfin topical solution (F-C) after 1 hour skin contact.

FIG. 4 shows representative images of upper back sebaceous glands containing Lemuteporfin-related fluorescence for different subjects in Cohort 2 from Example 9 following skin preparation and topical application of LTS at 0.1%. The upper four fluorescence images are from sites pretreated with infrared red (IR) heat followed by LTS at 0.1%. The lower four images are from skin sites dosed with LTS at 0.1% for 60 minutes without any skin pretreatment.

### DETAILED DESCRIPTION OF THE INVENTION

### Overview

The invention provides pharmaceutical compositions comprising photosensitizers, and methods of using the formulated photosensitizers to perform photodynamic therapy (PDT) for the treatment of dermatological disorders such as acne vulgaris and other hyperactive sebaceous gland disorders.

In order to perform PDT for sebaceous gland disorders, it is necessary to deliver photosensitizer into sebaceous glands. We observed that a previously known ointment formulation of the photosensitizer drug lemuteporfin, similar to that described in WO 03/039597, when applied to the skin of mice, was effective in localizing the photosensitizer to the sebaceous glands of this species. However, the same formulation was not generally as effective in localizing the drug to the sebaceous glands of humans. Therefore, we sought improved formulations that, when applied to human skin, would be capable of delivering an increased amount of a photosensitizer drug to sebaceous glands, preferably in a decreased amount of time.

Unexpectedly, we found that formulations of photosensitizer in the form of a liquid solution, without the addition of substantial amounts of viscosity modifying agents, such as thickeners, gelling agents, waxes, etc., were more effective than formulations such as gels, ointments, lotions, creams, etc. We discovered that addition of gelling agents such as hydroxy-propyl cellulose or ethyl cellulose in substantial amounts actually rendered the formulations relatively less capable of delivering photosensitizer to the sebaceous gland of either mice or humans. Such viscosity-modifying agents are frequently used in conventional topical therapies, and are considered generally useful in stabilizing supersaturated solution because they act as anti-nucleating agents.

We found that the most effective solution formulations that we developed contained concentrations of photosensitizer drugs that were approaching, and preferably exceeding the solubility of the drug in the formulations. Very surprisingly, we found solutions of green porphyrins such as lemuteporfin formulated above their solubility (supersaturated solutions) to be stable upon storage for up to 4 hours, even without the addition of anti-nucleating or gelling agents (for example polymers such as hydroxyl alkyl celluloses like hydroxypropyl methylcellulose (HPMC), hydroxypropyl cellulose (HPC), polyvinylpyrrolidone (PVP) and polyacrylic acid) that are typically used in the art to prevent precipitate from forming in a supersaturated solution. For example, the solubility of lemuteporfin in certain pharmaceutical formulations of the invention described herein ranges from about 0.025% to about 0.037% depending on whether surfactants are added. To achieve a concentration in the final formulation in the range of 0.05 to 0.2%, (which we have determined to be in the effective concentration range for performing PDT), a supersaturated solution is desirable. The unexpected stability of such supersaturated solutions for periods of time exceeding 4 hours was an important discovery in view of our observation (herein below) that the presence of polymers typically used in the art as anti-nucleating agents to prevent the precipitation of active ingredients from supersaturated solutions interfered with the localization of lemuteporfin to sebaceous glands. Hence the formulations described herein allow a relatively high concentration of lemuteporfin to be used, while maintaining lemuteporfin in solution for an amount of time that is therapeutically and commercially useful.

### Photosensitizer Formulations

As used herein the term “excipient” means the component(s) of a drug product other than the active pharmaceutical ingredient (API), including pharmaceutically acceptable diluents, vehicles, carriers, solvents, preservatives, antioxidants, viscosity modifying agents or combinations thereof.

As used herein, the term “solvent” means a pharmaceutically acceptable liquid solvent capable of dissolving a photosensitizer.

As used herein, the term “supersaturated” or “supersaturated solution” means, with respect to a photosensitizer, that the amount of photosensitizer dissolved in a solution exceeds the equilibrium solubility at a given temperature, usually ambient temperature or 20° C. unless otherwise indicated.

As used herein, the term “solubility” or “saturation solubility” means, with respect to a photosensitizer, the amount of the photosensitizer that can be dissolved in a given solvent at a given temperature at equilibrium, usually ambient temperature or 20° C. unless otherwise indicated.

In one aspect, the invention includes and provides a pharmaceutical composition useful for localizing a photosensitizer to a sebaceous gland, comprising a photosensitzer component and an excipient component in a solution, wherein the concentration of the photosensitizer in the solution is supersaturating, and wherein the photosensitizer does not precipitate out of solution to a pharmaceutically unacceptable degree after the solution is made. Without being bound by theory, it is thought that such supersaturated solutions are highly effective delivery systems for large molecules like lemuteporfin because the thermodynamic activity of the photosensitizer in the vehicle being at its highest and the resulting high concentration gradient being further increased by the evaporation of some of the volatile formulation components, lemuteprofin effectively partitions into the sebum, the waxy/oily mixture secreted by sebaceous glands, and the living cells (sebocytes) which comprise the PSU and sebaceous glands.

The invention further includes and provides a pharmaceutical composition comprising a solubilized photosensitizer and optionally, other excipients, wherein the concentration of photosensitizer in the composition exceeds the saturation solubility of the photosensitizer in the composition.

The invention also includes and provides a composition useful for topical delivery of a photosensitizer comprising a photosensitizer, one or more solvents and optionally one or more pharmaceutically acceptable excipients, wherein the composition has a viscosity of less than 50 centipoise (cps) at 20° C. Such a composition contains no (or very low amounts of) viscosity-modifiying agents, and may be supersaturated or not.

The photosensitizer component in the compositions may be present at concentrations ranging from about 0.001% to about 5% (w/w) depending on the type of photosensitizer chosen, its potency and its solubility. Typically, the photosensitizer component is present at concentrations ranging from about 0.01% to about 1.0%. For green porphyrins, such as lemuteporfin, preferred concentrations may range from 0.025% to about 0.5%, such as 0.025%, 0.05%, 0.075%, 0.1%, 0.125%, 0.15%, 0.175%, 0.2%, 0.225%, 0.25%, 0.3%, 0.4% or 0.5%. Concentrations of lemuteporfin in the range of 0.05% to 0.2% are preferred.

The excipient component in the compositions typically includes one or more solvents for the photosensitizer, such as benzyl alcohol (a preferred solvent for green porphyrins such as lemuteporfin), DGME (diethylene glycol monoethyl ether) or isopropyl alcohol. In some preferred embodiments, benzyl alcohol may be present in concentrations (w/w) ranging from about 1% to about 20%, or about 5% to about 15%, such as 5%, 6%, 7%, 8%, 9%, 10%, 11%, 12%, 13%, 14% or 15%. In one embodiment, benzyl alcohol is present at about 10%. In some embodiments DGME may be present in the excipient component in concentrations (w/w) ranging from about 5% to about 50%, from about 10% to about 40%, or from about 15% to about 35% such as 27%, 28%, 29%, 30%, 31%, 32%, 33%, 34%, 35% and 36%. In one embodiment, DGME is present at about 32%. In some embodiments, isopropyl alcohol may be present in the excipient component in concentrations (w/w) ranging from about 30% to about 85%, from about 40% to about 70%, from about 50% to about 60%. In some embodiments, isopropyl alcohol is present at 40%, 41%, 42%, 43%, 44%, 45%, 46%, 47%, 48%, 49%, 50%, 51%, 52%, 53%, 54%, 55%, 56%, 57%, 58%, 59%, or 60%. In one embodiment, isopropyl alcohol is present at about 49%.

In some embodiments, oleyl alcohol may be present in the excipient component at concentrations (w/w) ranging from 0% to about 6%, or about 2% to 5%. In one embodiment, oleyl alcohol is present at 5%. In some embodiments, polysorbate 80 may be present in the excipient component in concentrations ranging from 0% to about 1%, or about 0.25% to about 0.75%. In one embodiment, polysorbate 80 is present at 0.5%. In some embodiments, methyl salicylate is present in the excipient component in concentrations (w/w) ranging from 0% to about 2%, about 0.5% to about 1.5% or about 0.075% to about 1.25%. In one embodiment, methyl salicylate is present at about 1.0%. In some embodiments, menthol is present in the excipient component in concentrations (w/w) ranging from 0% to about 6%, about 1% to about 5% or about 2% to about 3%. In one embodiment, menthol is present at 2.5%.

Other solvents and excipients for photosensitizers may also include DMSO (dimethylsulfoxide), polyethylene glycol (PEG), PEG derivatives, glycol ethers, propylene glycol, polysorbates (e.g., Tween), fatty alcohols, aromatic alcohols, glycerols, oils, surfactants, glucosides, thiethylene glycol, tetraethylene glycol, pentaethylene glycol, hexaethylene glycol, septathylene glycol, octaethylene glycol, propylene glycol, propylene glycol mono- and di-esters of fats and fatty acids (e.g., propylene glycol monocaprylate, propylene glychol monolaurate), glycerol, mineral oil, lanolin, petrolatum or other petroleum products suitable for application to the skin, macrogols, macrogolglycerides or polyethylene glycol glycerides and fatty esters (e.g., stearoyl macrogolglycerides, oleoyl macrogolglycerides, lauroyl macrogolglycerides, linoleoyl macrogolglycerides), ethoxylated castor oil (e.g., Cremophor—a polyoxyl hydrogenated castor oil), C6-C30 triglycerides, natural oils, glucosides (e.g., cetearl glucosides and surfactants.

In some embodiments, the formulation composition need not contain substantial amounts of viscosity enhancing agents such as thickeners, gelling agents, etc. Such formulation compositions have a viscosity of less than 50 centipoise (cps) at 20° C. If needed or desired, the formulation compositions can be thickened by the addition of such viscosity enhancing agents as high MW polyethylene glycols, celluloses (such as hydroxypropyl cellulose or ethyl cellulose) acrylic acid-based polymers (carbopol polymers or carbomers), polymers of acrylic acid crosslinked with allyl sucrose or allylpentaerythrritol (carbopol homepolymers) polymers of acrylic acid modified by long chain (C10-C30) alkyl acrylates and crosslinked with allylpentaerythritol (carbopol copolymers), poloxamers (also known as pluronics; block polymers e.g., Poloxamer 124, 186, 237, 338, 407 etc), waxes (paraffin, glyceryl monostearate, diethylene glycol monostearate, propylene glycol monostearate, ethylene glycol monostearate, glycol stearate), hard fats (e.g., saturated C8-C18 fatty acid glycerides), xanthum gum, polyvinyl alcohol, solid alcohols, or mixtures thereof. However, as noted above, care must be taken when using viscosity modifying agents to ensure that they are not used in amounts that will interfere with delivery of the photosensitizers to sebaceous glands. In preferred embodiments exemplified herein, it is desirable not to add any viscosity modifying agents.

A supersaturated formulation of photosensitizer can be made in a number of ways. In one embodiment, a photosensitizer is dissolved in a good solvent for the photosensitizer (with or without heating), and then other excipients, in which the photosensitizer is less soluble, are added in. In another embodiment, a suspension of photosensitizer and solvent(s) and optionally other excipients can be heated until an amount of photosensitizer exceeding the solubility in the solvent(s) has been completely dissolved. In another embodiment, a photosensitizer is added below saturation solubility to one or more solvents(s) having one or more volatile components, such as ethanol, water, propanol, isopropanol or other volatile liquids known in the art. The volatile components evaporate to create a supersaturated condition in the less volatile components. For example, a non-saturated photosensitizer formulation for the treatment of acne can be prepared in excipients comprising volatile components. When the photosensitizer formulation is applied to the skin of a subject, some of the volatile components evaporate, creating a supersaturated solution in situ. In yet another embodiment, a supersaturated solution is prepared in excipients with one or more volatile components, and then further supersaturation occurs when the solution is applied to the skin of a subject as the volatile components evaporate.

### Long Term Stability of Formulations

We have found that supersaturated solutions of lemuteporfin are physically stable (i.e. lemuteporfin doesn't begin to precipitate out of solution) for at least 4 hours. If the desired concentration of the photosensitizer in the formulation composition exceeds the saturation solubility, and long term stability/shelf life (for example, 1-2 years) of the composition is desired then it may advantageous to provide a two-component formulation (or a multi-component formulation) where the components may be stored separately, and mixed prior to use.

Hence in another embodiment, a supersaturated solution is prepared by mixing a solution containing the photosensitizer component with a second solution comprising the excipient component, in which the solubility of the photosensitizer is lower. This aspect of the invention provides a pharmaceutical composition useful for localizing a photosensitizer to a sebaceous gland comprising a photosensitizing component comprising a photosensitizer, and associated therewith but separate therefrom, an excipient component, wherein the photosensitizer is present in an amount sufficient to form, on mixing, a supersaturated solution thereof and wherein the photosensitizer does not precipitate out of solution to a pharmaceutically unacceptable degree for a period of at least four hours once the photosensitizing component and the excipient component are mixed. Preferably, the two components are miscible, and thus may be easily combined by gentle shaking or stirring.

In a related aspect, the invention further provides a two-component pharmaceutical composition comprising two liquid phases, wherein at least one of the liquid phases comprises a photosensitizer dissolved therein, the two liquid phases are miscible, and the first liquid phase and the second liquid phase have a different solubilities of the photosensitizer, and wherein the concentration of the photosensitizer in each liquid phase is such that, upon combination of the two liquid phases, the total photosensitizer concentration in the liquid mixture is greater than the solubility of the photosensitizer in that liquid mixture, and the resulting liquid mixture is supersaturated with the photosensitizer. In an alternative embodiment, the photosensitizer is provided as a solid phase, rather than as a liquid solution. The photosensitizer solid is dissolved in a solvent prior to, or simultaneously with, mixing of the photosensitizer with the second liquid phase. The solid photosensitizer may be made amorphous or micronized to decrease the time to dissolution.

In some embodiments, the photosensitizing component comprises lemuteporfin dissolved in benzyl alcohol, with or without DGME. In some embodiments, the excipient component comprises DGME and isopropyl alcohol. In some embodiments the excipient component additionally comprises oleyl alcohol, menthol, methyl salicylate, or polysorbate 80. The concentrations of the elements of the photosensitizing component and the excipient component are adjusted so that the when the two components are combined, the final concentrations of the elements are in the concentration ranges provided above for lemuteporfin, benzyl alcohol, DGME, isopropyanol, oleyl alcohol, menthol, methyl salicylate, and polysorbate 80.

The concentration of photosensitizer in the photosensitizing component may range from the above the saturation solubility in the solvent downward. For a photosensitizing component comprising lemuteporfin dissolved in benzyl alcohol, the solubility is in the range of about 1.0% (w/w) to 2.5% w/w. In one embodiment, a photosensitizing component comprises a 1% w/w solution of lemuteporfin in benzyl alcohol, and prior to use it is mixed with an excipient component at a ratio of approximately 1 in 10 to give a final concentration of lemuteporfin in the formulation composition of about 0.1% w/w. In another embodiment, the photosensitizing component comprises a 2% solution of lemuteporfin in benzyl alcohol, and prior to use it is mixed with an excipient component at a ratio of approximately 1 in 10 to give a final concentration of lemuteporfin in the formulation composition of about 0.2% w/w. (A similar final product could also be obtained by mixing a photosensitizing component comprising a 1% solution of lemuteporfin with an excipient component at a ratio of 1 in 5.) It can thus be seen that the concentrations in the two components can be adjusted and manipulated to give the desired final concentrations of photosensitizer and excipients in the formulation to be used in PDT. Exemplary methods and compositions for some two-component formulations of the invention are given in the examples below.

In one aspect, the invention provides a method comprising the steps of
(a) providing a photosensitizing component comprising a photosensitizer dissolved in a solvent;
(b) providing an excipient component miscible with the photosensitizing component; and
(c) mixing an amount of the photosensitizing component with an amount of the excipient component to provide a mixed solution,
wherein the mixed solution is supersaturated with the photosensitizer.

In order to be useful clinically, the photosensitizer should not precipitate out of the pharmaceutical composition until it is applied to a subject. Preferably, the photosensitizer does not precipitate out of the pharmaceutical composition for at least about 30 seconds, about 1 minute, about 5 minutes, about 15 minutes, about 30 minutes, about 45 minutes or about an hour after the photosensitizing component is mixed with the excipient component. In other embodiments the photosensitizer does not precipitate out of the pharmaceutical composition for at least 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, or at least about 12 hours after the photosensitizing component is mixed with the excipient component. In some embodiments, the photosensitizer does not precipitate out of the pharmaceutical composition for up to at least about 16 hours, at least about 24 hours, at least about 48 hours, at least about 3 days, at least about 5 days, at least about 7 days, at least about 9 days, at least about 11 days, at least about 14 days, at least about 3 weeks, or at least about 4 weeks after the photosensitizing component is mixed with the excipient component. In other embodiments, the photosensitizer may remain dissolved for at least about 2 months, at least about 3 months, at least about 4 months, at least about 5 months or at least about 6 months after the photosensitizing component is mixed with the excipient component. In yet another embodiment, the photosensitizer may remain dissolved for at least about one year or at least about 2 years after the photosensitizing component is mixed with the excipient component.

In order to determine the time at which the photosensitizer may begin to precipitate out of a given pharmaceutical composition of the invention, and hence how long the composition may be kept before use, the compositions may be tested as follows. Samples of the compositions are taken at various time points after combining the photosensitizing component and the excipient component. Half of the samples are filtered to remove any precipitates, for example through a 0.22 μm filter. The filtered solutions are analyzed, for example, using HPLC, for the content or concentration of photosensitizer. If the solution is stable, and no photosensitizer has precipitated out, then the concentration of photosensitizer in the filtered solution should be roughly the same as the concentration of photosensitizer in the unfiltered solution, within experimental error. (This method was carried out in Example 10 to demonstrate that the stability of lemuteporfin in a formulation of the invention is at least 4 hours.) If the concentration of photosensitizer in the filtered and unfiltered samples is not roughly the same within experimental error, it may be considered that precipitation to a pharmaceutically unacceptable degree has occurred.

The components of the pharmaceutical composition should be mixed and then applied to the subject within the time period that the photosensitizer remains dissolved in the composition. In some embodiments, the components are combined within about 1 minute to about 24 hours of use. In one embodiment, the components are combined immediately prior to use. In another embodiment, the components are combined within about 30 seconds, about 1 minute, about 5 minutes, about 15 minutes, about 30 minutes, about 45 minutes or about an hour of use. In other embodiments the components are combined within about 1 hour to about 12 hours of use, such as within about 1, about 2, about 3, about 4, about 5, about 6, about 7, about 8, about 9, about 10, about 11, about 12 hours of use. In some embodiments, the components are combined within about 12 to about 24 hours of use, such as within about 12, about 13, about 14, about 15, about 16, about 17, about 18, about 19, about 20, about 21, about 22, about 23 or about 24 hours of use. In some embodiments, the components are combined within about 3 to 4 hours of use.

In another aspect, the invention also includes and provides a kit comprising a first container containing a photosensitizing component comprising a photosensitizer, and one or more containers containing excipient component(s) miscible with the solvents in the first container, and a set of instructions for combining the contents of the containers, topically applying the combined contents to the skin of a subject, and performing PDT for the treatment of one or more skin disorders. In one embodiment the containers are physically separate, for example, two or more vials. In another embodiment, the photosensitizing component and the excipient component(s) are packaged in a single container having two or more chambers that allow the components to be physically segregated from each other initially, and a release system to allow contact between chambers.

### Photosensitizers

As used herein “photosensitizer” or “photosensitizing agent” or “photosensitizing drug” means a chemical compound that absorbs electromagnetic radiation, most commonly in the visible spectrum, and releases it as another form of energy, most commonly as reactive oxygen species and/or as thermal energy. Preferably, the compound is nontoxic to humans or is capable of being formulated in a nontoxic composition. Preferably, the chemical compound produced upon photodegradation is also nontoxic. Hydrophobic and lipophilic photosensitizers tend to be especially useful for use in the compositions and methods of the invention because they may be more effective at partitioning into and diffusing through sebum and localizing in sebaceous glands.

A particularly potent group of photosensitizers is known as the green porphyrins, which are described in detail in U.S. Pat. No. 5,171,749, which is incorporated herein by reference in its entirety. The term “green porphyrins” refers to porphyrin derivatives obtained by reacting a porphyrin nucleus with an alkyne in a Diels-Alder type reaction to obtain a mono-hydrobenzoporphyrin. Such resultant macropyrrolic compounds are called benzoporphyrin derivatives (BPDs), which are synthetic chlorin-like porphyrins with various structural analogues and shown in U.S. Pat. No. 5,171,749.

Typically, green porphyrins are selected from a group of tetrapyrrolic porphyrin derivatives obtained by Diels-Alder reactions of acetylene derivatives with protoporphyrin under conditions that promote reaction at only one of the two available conjugated, nonaromatic diene structures present in the protoporphyrin-IX ring systems (rings A and B). Metallated forms of a green porphyrin, in which a metal cation replaces one or two hydrogen's in the center of the ring system, may also be used in the practice of the disclosed compositions and methods.

The preparation of green porphyrin compounds useful in this disclosure in described in detail in U.S. Pat. No. 5,095,030 which is incorporated herein in its entirety. Non-limiting examples of green porphyrins include benzoporphyrin diester di-acid (BPD-DA), mono-acid ring A (BPD-MA, also known as verteporfin), mono-acid ring B (BPD-MB), or mixtures thereof. These compounds absorb light of about 692 nm wavelength which has good tissue penetration properties. Particular useful for use herein are the group of green porphyrins known as ethylene glycol esters as set forth in U.S. Pat. No. 5,929,105. The compound referred to therein as A-EA6 is also known by the generic name lemuteporfin, is a highly preferred photosensitizer and has the following chemical structure:

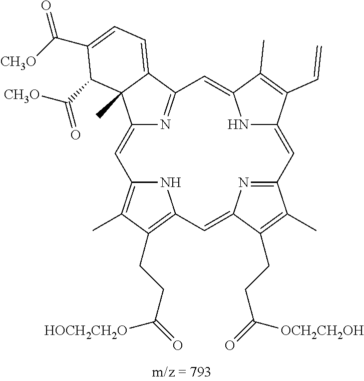

Additionally, the photosensitizers may be conjugated to various ligands to facilitate targeting to sebaceous glands or components thereof. These ligands include receptor-specific peptides and/or ligands as well as immunoglobulins and fragments thereof. Non-limiting ligands include antibodies in general and monoclonal antibodies, as well as immunologically reactive fragments of both.

Additional examples of green porphyrin photosensitizers include, but are not limited to, the green proprhyins disclosed in U.S. Pat. Nos. 5,283,255, 4,920,143, 4,883,790, 5,095,030 and 5,171,749, and green porphyrin derivatives discussed in U.S. Pat. Nos. 5,880,145 and 5,990,149. Several structures of typical green porphyrins are shown in the above cited patents, which also provide details for the production of the compounds.

There are a variety of other synthetic and naturally occurring photosensitizers that may be used, including, but not limited to, pro-drugs such as the pro-porphyrin %-aminolevulinic acid 5 ALA and derivatives thereof, porphyrins and porphyrin derivatives, e.g., chlorines, bacteriochlorins, isobacyteriochlorins, phthalocyanine and napththalocyanines and other tetra- and poly-macrocyclic compounds, and related compounds (e.g., pyropheophorbides, sapphyrins, and texaphrins) and metal complexes, (such as, but not limited to, tin, aluminum, zinc, lutetium). Use of tetrahydrochlorines, purpurins, porphycenes and phenothiaziniums is also contemplated. Other suitable photosensitizers include bacteriochlorophyl derivatives such as those described in WO 97/1981, WO 99/45382 and WO 01/40232. One bacteriochlorophyll is palladium-bacteriopheophorbide WST09 (Tookad™). A photosensitizer may be a proporphyrin or a porphryin, or a mixture thereof. Some examples of pre-drugs include aminolevulinic acid such a Levulan™ and aminolevulinic acid esters such as described in WO 02/10120 and available as Metvix™, Hexvix™ and Benzvis™. Some examples of di-hydro or tetra-hydro porphyrins are described in EP 0337,601 or WO 01/6650 and available as Foscan™ (temoporfin). Combinations of two or more photosensitizers may be used in the disclosed compositions and methods.

A nonexhaustive list of photosensitive chemicals may be found in Kreimer-Birnbaum, Sem. Hematol., 26:157-173 (1989), and in Redmond et al., Photoderm. Photobiol., 70(4):391-475 (1999), both of which are incorporated herein by reference.

### Light Energy Administration

Light of a suitable wavelength is applied to the skin to activate the photosensitizer. Preferably the light comprises a wavelength close to at least one of the absorption peaks of the photosensitizer. The absorption peaks differ for different photosensitizers. For example, lemuteporfin has an absorption peak at about 689 nm, and so, when lemuteporfin is the photosensitizer, the wavelength of light is preferably at or close of about 689 nm. The photosensitizer ALA-methyl ester (Metvix™) has an absorption peak at 635 nm and so the activation energy used is preferably at or close to 635 nm. The photosensitizer ALA (available under the trade name Levulan™) has absorption peaks at 417 nm and 630 nm so the activation energy used is preferably at or close to 417 and/or 630 nm.

The activation or light energy may be provided by any suitable means. Generally, the activation energy is provided by a visible light source. Light energy sources may include, but are not limited to, lasers, light emitting diodes (LED), incandescent lamps, standard fluorescent lamps, U.V. lamps or combinations thereof. Preferred light sources are light emitting diodes.

Commercially available light sources include CureLight™ (available from Photocure ASA, Oslo, Norway), BLU-U™ (available from DUSA Pharmaceuticals, Wilmington Mass., USA), PDT Laser (available from Diomed, Andover, Mass., USA), Ceralas™ (available from Biolitec AG, Jena, Germany), Omnilux PDT™ (available from PhotoTherapeutics Ltd., Birmingham, UK), and Q-Beam™ & Quantamed™ (Quantum Devices Inc., Barneveld, Wis., USA.)

In some embodiments, light is at least in part supplied by light emitting diodes (LEDs). For irradiating a contoured surface such as the face, it may be convenient to use a light source that is configured to follow the contour such as that described in U.S. Pat. No. 7,723,910. PDT for the treatment of acne can be combined with Blu-light Phototherapy in some embodiments of the invention. Therefore some embodiments include light being delivered by an LED device that supplies both red (e.g., 600-750 nm) and blue light (e.g., 390-450 nm). In some cases, a device supplies light at about 420 nm and at about 690 nm.

The dose of light or activation energy administered during a PDT treatment can vary according to the potency of the photosensitizer chosen. For photosensitizers of high potency, such as green porphyrins, the dosage of light is in the range of about 5 to about 400 J/cm², or more preferably in the range of about 25 to about 300 J/cm², as non-limiting examples. In some embodiments, the light dose used in PDT treatment is in the range of about 25 to about 50 J/cm², about 50 to about 100 J/cm², about 100 to about 150 J/cm², about 150 to about 200 J/cm², about 200 to about 250 J/cm², about 250 to about 300 J/cm², about 300 to about 350 J/cm², about 350 to about 400 J/cm², about 400 to 450 J/cm², about 450 to about 500 J/cm², about 500 to about 550 J/cm², or about 550 to 600 J/cm². Other non-limiting examples of light doses include doses of about 25, about 50, about 75, about 100, about 125, about 150, about 175, about 200, about 250 or about 300 J/cm².

The total light dose depends upon the intensity of the radiation source (also known as the fluence rate or irradiance) and the time of irradiation. Once the total dose of radiation is chosen, the fluence rate can be adjusted so that the treatment can be completed in a reasonable period of time. The period of irradiation or light exposure typically lasts from about 10 seconds to about 4 hours. For green porphyrins such as lemuteporfin, the light exposure typically lasts between 1 minute and 2 hours, more preferably between about 5 minutes and about 60 minutes. Some exemplary irradiation times are about 1, about 5, about 10, about 15, about 25, about 30, about 35, about 40, about 45, about 50, or about 55 or about 60 minutes.

The intensity of the energy or light source is generally below 600 mW/cm². Irradiances between about 10 and 500 mW/cm² are preferred, and even more preferably between about 25 and about 100 mW/cm². In some embodiments, the irradiance is 50 mW/cm². In other embodiments, the irradiance is 80 mW/cm². In other embodiments, the light dose is varied between 37.5 J/cm² and 150 J/cm² by varying the time of irradiation at a fixed fluence rate of 80 mW/cm² between 7 min. 49 sec. to 31 min 15 sec.

### PDT Treatment of Acne and Other Hyperactive Sebaceous Gland Conditions

The invention also includes and provides methods for treating a hyperactive sebaceous gland disorder in an affected area of the skin of a subject in need thereof, comprising topically applying a therapeutically effective amount of a photosensitizer composition of the invention to the affected area of the skin of the subject, allowing sufficient time for at least some of the photosensitizer to localize in the sebaceous glands, and exposing the skin of the subject to light energy at a wavelength capable of activating the photosensitizer. In some embodiments, the hyperactive sebaceous gland disorder is acne (including acne vulgaris), seborrhea (or oily skin), seborrheic dermatitis, hidradenitis suppurativa (acne inversa), and sebaceous gland hyperplasia. In some embodiments, the subjects have both acne and oily skin.

The invention also includes and provides methods for reducing sebum production by sebaceous glands of a subject in need thereof, comprising topically applying a therapeutically effective amount of a photosensitizer composition of the invention to the affected skin of a subject in need of treatment, allowing sufficient time for at least some of the photosensitizer to localize in the sebaceous glands, and exposing the skin of the subject to light energy at a wavelength capable of activating the photosensitizer, whereby the sebum excretion rate of the subject is reduced.

The invention also includes and provides methods of treating acne in a subject in need thereof, comprising topically applying a therapeutically effective amount of a photosensitizer composition of the invention, allowing sufficient time for at least some of the photosensitizer to localize in the sebaceous glands of the subject, and exposing the skin of the subject to light energy at a wavelength capable of activating the photosensitizer.

The invention also includes and provides methods for ablating sebocytes in a subject afflicted with a hyperactive sebaceous gland disorder such as acne, comprising the steps of delivering a therapeutically effective amount of a photosensitizer to the sebocytes of the subject, allowing sufficient time for the photosensitizer to localize in the sebocytes, and exposing the sebocytes to light energy at a wavelength capable of activating the photosensitizer.

Conditions that may be treated include any condition for which a topical formulation of a photosensitizer is suitable. Non-limiting examples include skin conditions such as dermatitis, psoriasis, malignant and pre-malignant skin lesions, actinic keratosis, and hyperactive sebaceous gland disorders. Hyperactive sebaceous gland disorders include, without limitation, acne (including acne vulgaris), seborrhea (or oily skin), seborrheic dermatitis, hidradenitis, suppurativa, and sebaceous gland hyperplasia. Interior body cavities such as the mouth or uterus may also be treated. Any part of the body may be treated, but conditions such as acne and oily skin typically affect the face, chest and/or back.

For a PDT treatment, the skin is first preferably washed with an antibacterial cleanser and dried. The skin may be treated with dry heat (IR) until either the skin temp reaches 45 C or for a fixed time such as 20 min. This may enhance the penetration of photosensitizer into the sebaceous glands. Alternatively, the skin may also be treated with microderm abrasion. The skin may be degreased (e.g. using acetone or isopropyl alcohol) if necessary.

Once this skin surface has been cleansed and prepared, the chosen formulation of photosensitizer is applied to the affected area of a skin surface after the area has been thoroughly cleansed. The photosensitizer-containing formulation is left in contact with the skin for sufficient time to allow the photosensitizer to localize in the sebaceous glands of the subject. Generally the time of contact could be between about 1 minute and about 24 hours or longer, depending on the type and concentration of the photosensitizer in the formulation. Preferably, the formulation is in contact with the skin for about 1 to about 180 minutes if the photosensitizer is a green porphyrin such as lemuteporfin. Exemplary contact times are about 1, about 5, about 10, about 20, about 30, about 40, about 50, about 60, about 70, about 80, about 90, about 100, about 110, about 120, about 130, about 140, about 150, about 160, about 170 or about 180 minutes. Additional exemplary contact times are about 3.5, about 4, about 4.5, about 5, about 5.5, about 6, about 6.5, about 7, about 7.5 or about 8 hours. Excess formulation is then preferably removed with clean gauze or cloth moistened with lukewarm water. Irradiation is then applied as described above. It may be advisable to use a regimen of increasing light dose until the subject maximum tolerated dose (MTD) is determined. Pain at the site of irradiation or erythema following PDT are signs that the MTD has been exceeded. Thereafter, the person may be treated at the MTD.

The treatment may be repeated as many times as necessary to have a therapeutic effect. If repeated, the treatment frequency may vary. For example, the treatments could be daily, about every two days, about twice weekly, about weekly, about every two weeks, about twice monthly, about every four week, about monthly, about every six weeks, about every eight weeks, about every two months, about quarterly, about twice annually, or about annually, or other suitable time interval. A preferred treatment interval is every two weeks to every six months. Treatment can continue until the desired degree of improvement in the skin condition has occurred. For example, treatments may be repeated until the total number of acne lesions is reduced by about 5%, about 10%, about 15%, about 20%, about 25%, about 30%, about 40%, about 50%, about 60%, about 70%, about 80% or about 90% or more. To take another non-limiting example, treatments may be repeated until the sebum excretion rate has been reduced by about 5%, about 10%, about 15%, about 20%, about 25%, about 30%, about 40%, about 50%, about 60%, about 70%, about 80% or about 90% or more.

### Determining Efficacy of Treatment

The efficacy of the disclosed compositions and methods may be determined by any suitable means. In many cases, a simple decrease, reduction, or improvement in the sebaceous gland disorder or other skin disorder, as recognized by a skilled physician may be used to determine efficacy. Thus an improvement in a hyperactive sebaceous gland disorder, such as an improvement in a subject's acne, seborrhea, seborrheic dermatitis, hidradenitis suppurativa, or sebaceous gland hyperplasia, may be used as an indication of efficacy.

Taking acne as a non-limiting example, efficacy may be determined based upon quantitative and/or qualitative data. The total number of lesions can be assessed by predefining one or more test areas before commencement of treatment. Lesion counts (non-inflammatory, inflammatory and total, or open comedones, closed comedones, papules, pustules and nodules) are performed within the test area before and after treatment. Sizes of the lesions within the test area are also recorded. The test areas are also photographed. A number of test areas may be selected for each subject and the location of the test area may vary depending on the locale of the acne lesions of the subject. The test areas may be assessed within the first week, after one week, after two weeks, or after a month or two of the initial PDT treatment, or at other desired frequencies. A global assessment scale such as the 5-point Investigator Global Assessment (IGA) for acne vulgaris, as recommended by the FDA and shown in Table 1 may be used to measure efficacy.

TABLE 1
Investigator's Global Assessment (IGA) Scale

| Score | Description |
|---|---|
| 0 | No acne: No evidence of acne vulgaris |
| 1 | Minimal: Few non-inflammatory lesions |
| | (comedones) are present; a few inflammatory |
| | lesions (papules/pustules) may be present) |
| 2 | Mild: Several to many non-inflammatory |
| | lesions (comedones) are present; a few |
| | inflammatory lesions (comedones) are present |
| 3 | Moderate: Many non-inflammatory (comedones) |
| | and inflammatory (papules/pustules) are |
| | present; no nodulocystic lesions are present |
| 4 | Severe: Significant degree of inflammatory |
| | disease; papules/pustules are a predominant |
| | feature; a few nodulocystic lesions may be |
| | present; comedones may be present |

The efficacy of PDT for reducing sebum production may be measured by using SebuTape™, a product designed specifically for that purpose and available from CuDerm Corporation, Dallas, Tex., USA. Example 9 herein demonstrates how to use SebuTape to obtain an accurate measurement of sebum exudation. SebuTape measurements may be done within the first week, after one week, after two weeks, or after a month or two of the initial PDT treatment, or at other desired frequencies. The efficacy of PDT for reducing the number of sebaceous glands may be measured by taking biopsies following PDT treatment, and using histology with Oil Red O staining to determine the total number of PSU (hair follicles structures with or without sebaceous glands) in an image followed by a count of the number of lipid-staining (sebaceous gland containing) staining PSU. This procedure is described in Example 3 herein.

### EXAMPLES

### Example 1

### Solubility of Lemuteporfin in Various Solvents

The solubility of lemuteporfin in various solvent compositions is shown in the last column of Table 2. All values were obtained analytically by HPLC analysis.

Solubility results for lemuteporfin indicated maximum solubility in solvent-based formulations containing primarily benzyl alcohol. The solubility of lemuteporfin in benzyl alcohol is approximately 2.5% w/w. The addition of other solvents reduced solubility by approximately the amount of the new solvent introduced. Diethylene glycol monoethyl ether (DGME) is about 20% as efficient at dissolving lemuteporfin as benzyl alcohol.

TABLE 2
Solubility Of Lemuteporfin In Various Solvents

| | Benzyl | Oleyl | Oleic | Propylene | Ethanol | Isopropyl | % w/w |
|---|---|---|---|---|---|---|---|
| DGME* | Alcohol | Alcohol | Acid | Glycol | (190) | Myristate | Lemuteporfin |
| 65 | — | 35 | — | — | — | — | 0.430 |
| 65 | — | 17.5 | 17.5 | — | — | — | 0.271 |
| 50 | 15 | — | 35 | — | — | — | 0.230 |
| 50 | 15 | 35 | — | — | — | — | 0.200 |
| 52 | 16 | 16 | 16 | — | — | — | 0.210 |
| — | 30 | — | — | — | 70 | — | 0.455 |
| — | 100 | — | — | — | — | — | 2.55 |
| — | 30 | 70 | — | — | — | — | 0.166 |
| 50 | 50 | — | — | — | — | — | 1.57 |
| — | 50 | — | — | 50 | — | — | 0.494 |
| — | 50 | — | — | — | 50 | — | 0.453 |
| — | 50 | 50 | — | — | — | — | 0.228 |
| — | 50 | — | 50 | — | — | — | 0.409 |
| — | 40 | — | — | — | — | 60 | 0.090 |
| 90 | — | 10 | — | — | — | — | 0.420 |
| 90 | — | 5 | 5 | — | — | — | 0.420 |
| 90 | — | — | — | — | — | 10 | 0.430 |
| — | 90 | 10 | — | — | — | — | 2.24 |
| — | 90 | — | 10 | — | — | — | 2.33 |
| — | 90 | 5 | 5 | — | — | — | 2.22 |
| — | 90 | — | — | — | — | 10 | 2.30 |
| 45 | 45 | 5 | 5 | — | — | — | 1.373 |

*values under solvent name are % w/w of solvent in solution composition

### Example 2

### The Effect of Viscosity-Enhancing Agents on Photosensitizer Formulations

This experiment was performed to assess the impact of increasing the viscosity of lemuteporfin formulations on the efficacy of PDT to ablate mouse sebaceous glands. Photosensitizer compositions were prepared with the components shown in Table 3 and applied onto shaved mouse flank skin for 30 minutes prior to exposure with 688 nm red light (50 J/cm2 or 100 J/cm2 delivered at a rate of 50 mW/cm2). Each treatment group consisted of 5 animals.

To assess sebaceous gland changes, mice were sacrificed 72 hours after PDT. Full-thickness skin from within the tattoo points on the PDT-treated right flank was carefully excised. The upper half of these tissue squares was placed in a plastic mold filled with “Neg 50” cryo embedding medium and frozen on liquid nitrogen. The lower half was preserved in formol acetic alcohol for 18 hours. The tissue was transferred to 70% alcohol until processed to wax by a standard in-house protocol. Formalin-fixed samples were subsequently stained with standard reagents (e.g., hematoxylin and eosin) to assess general histological changes within the tissue if required.

For sebaceous gland evaluations, frozen tissue samples were cut in 8 μm sections with a cryostat onto glass slides and immediately fixed in 10% buffered formalin. Three sets of 2 slides were cut from each block with the distance between sets of approximately 200 μm. One slide from each set was stained with Oil Red O and then cover-slipped with acrylic mounting medium and allowed to set. The second slide from each set was used as a “back-up” in the event that the first slide was damaged.

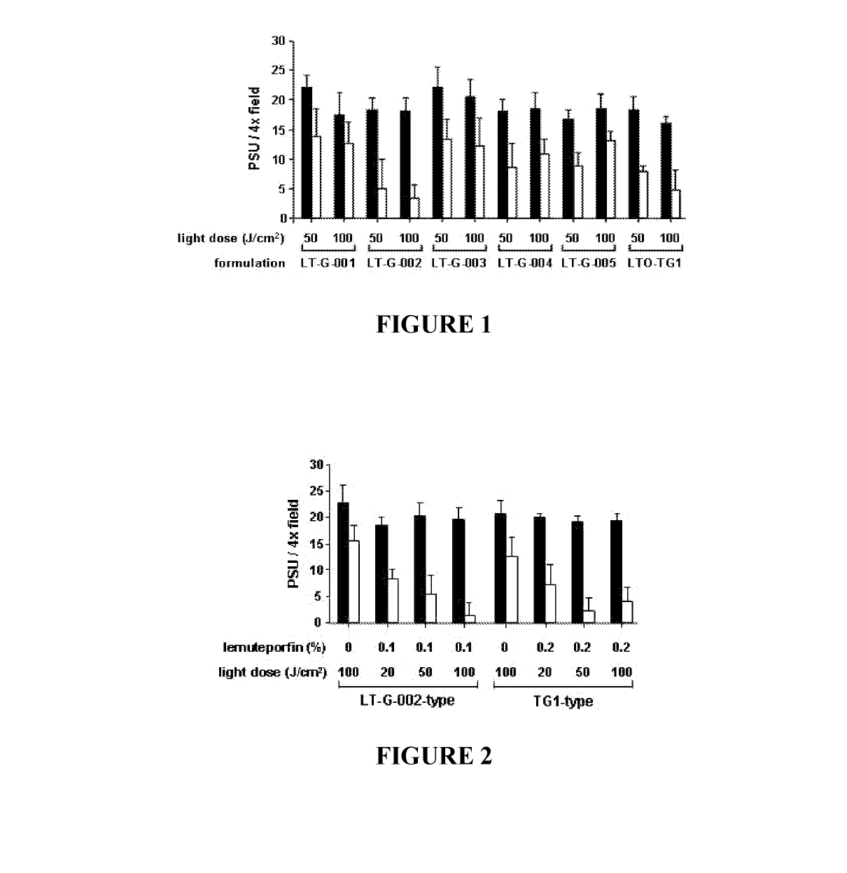

Images were taken of representative sections from each cross-section using a 4× objective mounted on an Olympus BX61 microscope fitted with a digital camera. Slides were assessed by counting the total number of PSU (hair follicles structures with or without sebaceous glands) in an image followed by a count of the number of lipid-staining (sebaceous gland containing) staining PSU. Slides were evaluated by two independent readers. The results are shown in FIG. 1.

TABLE 3
Lemuteporfin Formulation Components for LT(S) and LTO

| Component | | | | | | |
|---|---|---|---|---|---|---|
| (w/w %) | LT-G-001 | LT-G-002 | LT-G-003 | LT-G-004 | LT-G-005 | LTO-TG1 |
| lemuteporfin | 0.1 | 0.1 | 0.1 | 0.1 | 0.02 | 0.2 |
| benzyl alcohol | 5 | 5 | 5 | 5 | 5 | — |
| isopropyl alcohol | 48.9 | 48.9 | 48.9 | 48.9 | 48.9 | — |
| diethylene glycol | 32 | 32 | 32 | 32 | 32 | 20.0 |
| monomethyl ether | | | | | | |
| oleyl alcohol | 5 | 5 | 5 | 5 | 5 | 10.0 |
| methyl salicylate | 1 | 1 | 1 | 1 | 1 | — |
| polysorbate 80 | 0.5 | 0.5 | 0.5 | 0.5 | 0.5 | — |
| menthol | 2.5 | 2.5 | 2.5 | 2.5 | 2.5 | — |
| hydroxy-propyl | 4 | — | 2 | 4 | 4 | — |
| cellulose | | | | | | |
| ethyl cellulose | 1 | — | | | 1 | — |
| PEG400 | — | — | — | — | — | 53.8 |
| PEG3350 | — | — | — | — | — | 16.0 |

Due to the large number of mice required to test a matched-vehicle for each formulation, no control groups were included in this experiment. However, typically, for nai\ddot{v}e mouse flank skin 70-80% of PSU contained detectable Oil Red O-positive sebaceous glands. The most effective composition in producing the lowest number of PSU with Oil Red O-positive sebaceous glands was formulation LT-G-002 (FIG. 1). This formulation did not contain a viscosity modifying agent. On average, approximately 30% of PSU in flank skin treated with LT-G-002 and either light dose contained Oil Red O-positive glands. PDT with LTO-TG1 had a similar, but somewhat lower, reductive effect on sebaceous gland counts. In contrast, sebaceous gland counts for mice treated with PDT using Formulations LT-G-001, LT-G-003, LT-G-004 or LT-G-005 (all containing a viscosity enhancing agent that was either hydroxy-propyl cellulose, ethyl cellulose, or both) were not nearly as effective. We concluded that such viscosity enhancing agents may have prevented the localization of the lemuteporfin in sebaceous glands.

### Example 3

### Effect of Different Light Doses on PDT of Mouse Sebaceous Glands with a Lemuteporfin Composition Lacking a Viscosity Enhancing Agen

This experiment compared the effect of PDT with LT-G-002 to that of LTO-TG1 (which contained twice the amount of lemuteporfin) at three different red light doses. PDT with either lemuteporfin topical formulation affected sebaceous counts with reductive effects observed at red light doses of 20, 50 or 100 J/cm² as compared to the result obtained for mice treated with control vehicle and a red light dose of 100 J/cm² (FIG. 2). Greater effects on gland counts, with either lemuteporfin formulation, were produced with red light doses of 50 and 100 J/cm² than at 20 J/cm².

### Example 4

### Localization of Lemuteporfin in Human Hair Follicles and Sebaceous Glands

The model for localization of lemuteporfin in human skin utilized dermatomed human cadaver skin procured from Ohio Valley Tissue Bank, fresh (≦24 hours post-mortem) and human skin procured from NDRI (National Disease Research Interchange). This experiment compared a lemuteporfin topical solution (LTS) without a viscosity enhancing agent to a lemuteporfin topical ointment (LTO-TG1 from Example 2, Table 3). The LTS formulation included lemuteporfin, 0.1%, oleyl alcohol, 5%, benzyl alcohol 5%, DGME 32%, Vitamin E TPGS, 0.5%, menthol, 5%, and ethanol, 52% all w/w. The formulations were applied to the skin in a measured amount and left open to the air. The skin was maintained in contact with the formulations for the designated period of time (1 or 8 hours), biopsied, set in Neg-50 frozen tissue medium and then prepared for sectioning and fluorescence microscopy evaluation.

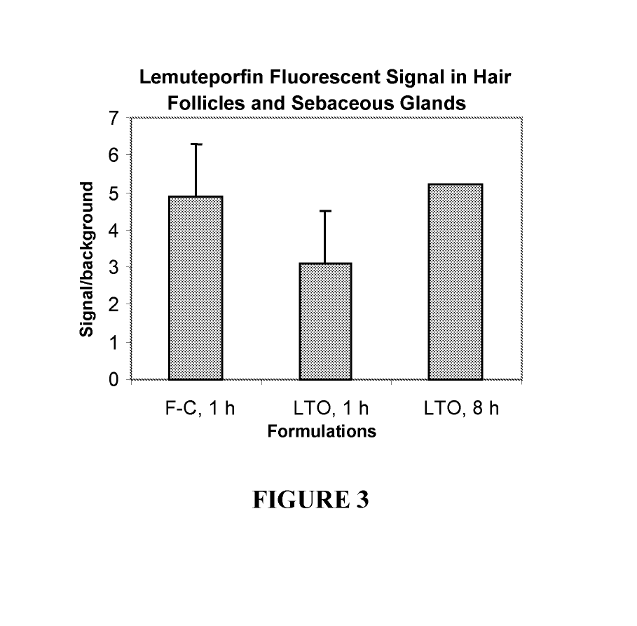

The tissue fluorescence results showed that the LTS formulation localized in human cadaver skin sebaceous glands within one hour to an extent that it required 8 hours for an LTO formulation containing twice the amount of lemuteporfin to achieve (FIG. 3). We therefore believe that a solution type of formulation provides more rapid delivery of lemuteporfin to human sebaceous glands than an ointment form. This is important in a clinical setting in which a subject must wait for a specified period of time between application of a photosensitizer-containing formulation and activation of the photosensitizer with light: a shorter period of time is better.

### Example 5

### Stability of LTS Photosensitizer Composition

A batch of lemuteporfin topical solution was prepared according to the formula in Table 4, dispensed into 5 ml vials, and maintained for stability testing. After three months, precipitation was observed in some vials. The precipitate was identified as lemuteporfin. An optimal delivery system for lemuteporfin contains a relatively high concentration of lemuteporfin, but also must contain components in which lemuteporfin is not readily soluble such as DGME (see Example 1). Thus it was necessary to implement a different approach to formulating lemuteporfin if long term storage is desired.

TABLE 4
A lemuteporfin topical solution (LTS)

| Formulation | Percentage |
|---|---|
| Component | w/w |
| Lemuteporfin | 0.1% |
| Oleyl Alcohol | 5% |
| Benzyl Alcohol | 10% |
| DGME | 32% |
| Polysorbate 80 | 0.5% |
| Menthol | 2.5% |
| Isopropanol | 48.9% |
| Methyl Salicylate | 1.0 |

### Example 6

### Solubility Studies of Formulated Lemuteporfin

LTS was prepared by adding lemuteporfin to the other components in Table 4 (already pre-mixed) at room temperature. The solution was stirred and samples were removed at various time points, and filtered to determine the amount of undissolved lemuteporfin. The results are presented in Table 5. The amount of lemuteporfin that dissolved was approximately 0.048%.

It was possible to manufacture a lemuteporfin topical solution 0.1% by dissolving the drug in DGME and benzyl alcohol at high temperature, approximately 75° C. The solution was then cooled to room temperature and the remaining LTS components were added and mixed to form a homogenous solution. Based on the solubility data, this manufacturing process resulted in a supersaturated solution.

TABLE 5
Lemuteporfin Solubility in
the Formulation Shown in Table 4

| Time point | Lemuteporfin content |
|---|---|
| (hours) | (% w/w) |
| 0.17 | 0.0241 |
| 2.47 | 0.0482 |
| 4.37 | 0.0453 |
| 6.37 | 0.0463 |
| 23.5 | 0.0370 |

Studies were conducted to determine the effect of certain of the LTS excipients on the solubility of lemuteporfin. Removing isopropyl alcohol from the solution system increased the solubility of lemuteporfin from approximately 0.03% to 0.07% w/w (data not shown). Polysorbate 80 increased the solubility from 0.027% to 0.037% w/w (data not shown).

### Example 7

### Two-Component Formulation System

To solve the problems of the solubility and long-term stability of lemuteporfin in an effective topical delivery formulation, we developed a two-component formulation system. The first component comprises lemuteporfin dissolved in a solvent in which it is highly soluble. The second component comprises the remainder of the LTS excipients. Some examples of LTS two-component formulations are shown in Tables 6 to 10.

The compositions described in Tables 6 to 10 were made as follows. The active (lemuteporfin-containing) and non-active solutions were manufactured in separate compounding vessels. A jacketed beaker connected to a water bath was set at 75° C. and was placed on a stir plate. The active solution was mixed while being heated for approximately 1 hour. After one hour of heating, the active solution was cooled to room temperature while continuing to mix.

The non-active excipients were weighed and transferred to a separate glass vessel. The excipients were mixed at room temperature for approximately 30-60 minutes.

Filling was performed using a Flexicon vial filler. Fill checks were performed and the average fill weight was within the 2% of the target fill weight. The non-active solutions were filled first followed by the active solution for each batch manufacture. After filling, all vials were labeled and then placed at 2-8° C.

TABLE 6
Batch A (0.1% w/w)

| Vial | Component | % w/w | actual weights (g) |
|---|---|---|---|
| 1 | Lemuteporfin | 0.1 | 0.5001 |
| | Diethylene glycol | 32 | 160.1939 |
| | monoethyl ether | | |
| | (DGME) | | |
| | Benzyl alcohol | 5 | 25.0829 |
| 2 | Isopropanol (IPA) | 53.9 | 269.5 |
| | Oleyl alcohol | 5 | 25.0595 |
| | Polysorbate 80 | 0.5 | 2.5141 |
| | Methyl salicylate | 1 | 5.0144 |
| | Menthol | 2.5 | 12.5040 |
| | Total weight | 100 | 500.3689 |

TABLE 7
Batch B (0.075% w/w)

| Vial | Component | % w/w | actual weights (g) |
|---|---|---|---|
| 1 | Lemuteporfin | 0.075 | 0.3751 |
| | Diethylene glycol | 32 | 160.0135 |
| | monoethyl ether | | |
| | (DGME) | | |
| | Benzyl alcohol | 5 | 25.0653 |
| 2 | Isopropanol (IPA) | 53.925 | 269.6259 |
| | Oleyl alcohol | 5 | 25.0114 |
| | Polysorbate 80 | 0.5 | 2.5017 |
| | Methyl salicylate | 1 | 5.0016 |
| | Menthol | 2.5 | 12.5091 |
| | Total weight | 100 | 500.1036 |

TABLE 8
Batch C (0.1% w/w) - Formulation LemuteporfinTK1

| | Target | | | | actual |
|---|---|---|---|---|---|
| | Fill | | | % | weights |
| Vial | Weight | | Component | w/w | (g) |
| 1 | 0.9 | g | Lemuteporfin | 0.1 | 1.0005 |
| | | | Benzyl alcohol | 10 | 100.1729 |
| 2 | 8.01 | g | Diethylene glycol | 32 | 320.1 |

| | | |
|---|---|---|
| monoethyl ether | | |
| (DGME) | | |
| Isopropanol (IPA) | 48.9 | 490.0 |
| Oleyl alcohol | 5 | 50 |
| Polysorbate 80 | 0.5 | 5 |
| Methyl salicylate | 1 | 10.1 |
| Menthol | 2.5 | 25.0 |
| Total weight | 100 | 1001.3734 |

TABLE 9
Batch D (0.075% w/w) - Formulation LemuteporfinTK2

| | Target | | | | actual |
|---|---|---|---|---|---|
| | Fill | | | % | weights |
| Vial | Weight | | Component | w/w | (g) |
| 1 | 0.9 | g | Lemuteporfin | 0.075 | 0.7507 |
| | | | Benzyl alcohol | 10 | 100.02 |
| 2 | 8.03 | g | Diethylene glycol | 32 | 320 |

| | | |
|---|---|---|
| monoethyl ether | | |
| (DGME) | | |
| Isopropanol (IPA) | 48.925 | 336.9 (9-2056) |
| | | 153.0 (9-2075) |
| | | Total: 489.9 |
| Oleyl alcohol | 5 | 50.1 |
| Polysorbate 80 | 0.5 | 5.1 |
| Methyl salicylate | 1 | 10.5 |
| Menthol | 2.5 | 25.1 |
| Total weight | 100 | 1001.4707 |

TABLE 10
Batch H (0.05% w/w) - Formulation LemuteporfinTK3

| | Target | | | | actual |
|---|---|---|---|---|---|
| | Fill | | | % | weights |
| Vial | Weight | | Component | w/w | (g) |
| 1 | 0.9 | g | Lemuteporfin | 0.05 | 0.5001 |
| | | | Benzyl alcohol | 10 | 100.3 |
| 2 | 8.0 | g | Diethylene glycol | 32 | 320.1 |

| | | |
|---|---|---|
| monoethyl ether | | |
| (DGME) | | |
| Isopropanol (IPA) | 48.95 | 489.9 |
| Oleyl alcohol | 5 | 50.0 |
| Polysorbate 80 | 0.5 | 5.1 |
| Methyl salicylate | 1 | 10.1 |
| Menthol | 2.5 | 25.1 |
| Total weight | 100 | 1001.1 |

### Example 8

### Lemuteporfin Drug Localization in Human Sebaceous Glands: Comparison of LTS (0.02%), LTS (0.1%) and LTO (0.2%).

Lemuteporfin sebaceous gland localization with the LTS formulation was studied in a human clinical study. The work was performed to evaluate two strengths (0.02%, 0.1%) of lemuteporfin topical solution (LTS) formulation for their potential to support the distribution of lemuteporfin-related fluorescence into sebaceous glands of the upper back of healthy subjects, either with or without prior skin preparation. An earlier generation formulation, lemuteporfin Topical Ointment (LTO) 0.2% under occlusion in combination with infrared (IR) heat skin preparation was tested in parallel as a control treatment since its sebaceous gland delivery properties had been previously studied. The composition of LTO had been determined to be non-optimal for delivery of lemuteporfin into human sebaceous glands. The safety and local tolerability of LTS, in combination with and without different skin preparation methods, was also evaluated in this study.

### Study Design

A partial-blind, sequential, randomized drug-localization study consisting of two cohorts of 10 healthy human subjects each (20 subjects total) was carried out under informed consent. Each of the 20 study subjects attended all scheduled visits and completed the study. The mean age of subjects was 24 years (range: 18-30 years). Eleven (55%) of the subjects were female. Cohorts 1 and 2 evaluated 2 different dose strengths of LTS, 0.02% w/w and 0.1% w/w, respectively. Each subject had four test sites (2 cm×2 cm) positioned on the upper back. Subjects received each of the four treatment regimens:
LTS without any skin preparation
LTS after skin preparation with micro-derm-abrasion (MDA)
LTS after skin preparation with dry heat from an IR heat device
LTO with plastic film occlusion after skin preparation with dry heat from IR device

Each formulation was allowed to remain in contact with the skin for approximately 60 minutes. Upon completion of the contact time, excess material was removed from the test sites using clean gauze dampened with lukewarm water and then a 4 mm punch biopsy was taken from each test site.

### Sebaceous Gland Fluorescence Analysis

Biopsies were placed in Neg-50 frozen section embedding medium and snap-frozen in liquid nitrogen. Samples were stored at −70° C. until shipped on dry ice to the histology laboratory with extensive experience in the required methodology. Tissue blocks were placed onto a chuck of a Microm EM500 Cryostat and then trimmed to expose the tissue area. Eight micron thick sections were cut onto microscope slides which were immediately covered with a glass cover slip adhered by Prolong Antifade (Molecular Probes) and stored in a light-opaque box at 4° C.

For each biopsy sample, approximately twenty slide sets were prepared. Each of these sets consisted of 3 slides. The first three sets were appraised for the absence/presence of sebaceous glands. Generally, the next five sets were omitted and the following three sets were assessed for the presence of sebaceous gland structures. This selection process continued until a total of nine sets with acceptable sebaceous gland presence were identified. However, if the last slide set had been evaluated without nomination of nine sets with adequate sebaceous gland representation, omitted sets then were examined in the sequence that they were prepared until nine sets were acquired. If nine sets were not obtainable from the biopsy sample, then the maximum available number of sets was ultimately evaluated.

Fluorescence microscopy was used to evaluate the distribution of lemuteporfin in the skin and to determine if there was specific accumulation of lemuteporfin in the sebaceous glands. Slides were viewed with a Zeiss Axiovert TV 100 microscope equipped with a monochromatic Photometrics 350 camera (Roper Scientific). The sections were initially viewed under bright field illumination to identify sections with sebaceous glands. Images were then taken with epi-fluorescence illumination appropriate for lemuteporfin (excitation 425 nm; emission 690 nm). The exposure for each fluorescent image was 5 seconds with a 5× lens objective covering a 2×2 mm area at this magnification. Each image was taken at 16-bit depth which results in 65500 shades of grey. This setting gives increased precision for fluorescence detection. The display range (i.e. contrast intensity) for all samples was set to a scale of 500-5000 using Image-Pro Plus software. In previous studies it was consistently observed that skin biopsy samples obtained from lemuteporfin-naïve skin exhibited no detectable fluorescence.

Biopsy sample images were appraised for the distribution of fluorescence within sebaceous glands examined by a panel of experienced evaluators who were blinded to the identity and origin of the samples. With group consensus, samples were deemed positive for sebaceous gland uptake of lemuteporfin if the fluorescence distinctly revealed general gland structure and/or outlined gland lobules with greater intensity than the surrounding tissues.

The non-parametric Chi-(X2)-square test was performed to reveal whether the observed differences in sebaceous gland lemuteporfin fluorescence results for the different treatments within each cohort were statistically significant.

### Results

In this drug-distribution study, the different skin preparation methods employed as well as application of the LTS/LTO formulations were generally well-tolerated. Edema was not observed at any test site. When localized skin erythema was observed it was primarily associated with the skin preparation procedures.

The sebaceous gland localization of lemuteporfin applied in different topical formulations was assessed using tissue fluorescence image analyses. lemuteporfin fluorescence signal was evident within hair follicles and sebaceous glands with the different test regimens although to various degrees. For all samples, there was no appreciable fluorescence signal in surrounding non-pilosebaceous structures. In some samples, strong lemuteporfin fluorescence was associated with plugs within the outer pore region of hair follicles. This circumstance produced a fluorescence flaring phenomenon which emanated into adjacent portions of these samples. Such observations were typically recorded as a negative result unless sufficiently prominent and separated sebaceous gland fluorescence was also present. Several sections exhibited drug fluorescence in the stratum corneum layer suggesting that some residual drug had remained on the skin surface.

For sections obtained from control (IR heat pretreatment plus 0.2% LTO with occlusion) sites, skin areas exposed to LTS at 0.1% combined with MDA or lower strength LTS (0.02%) with different pretreatments, approximately 20% of these slides had fluorescence signal evident within sebaceous glands (Table 11). Fluorescence image findings for the control sites (IR heat pretreatment plus 0.2% LTO) were similar for Cohorts 1 and 2 (19.2% and 19.1%, respectively) indicating the reproducibility of the treatment and analysis methodology. For subjects treated with LTS at 0.02% versus LTO at 0.2% under occlusion, there was no significant difference in the proportion of group samples with lemuteporfin-related sebaceous gland fluorescence as determined by the non-parametric Chi-square statistical test (X2 value=1.36, 3 degrees of freedom, P=0.715).

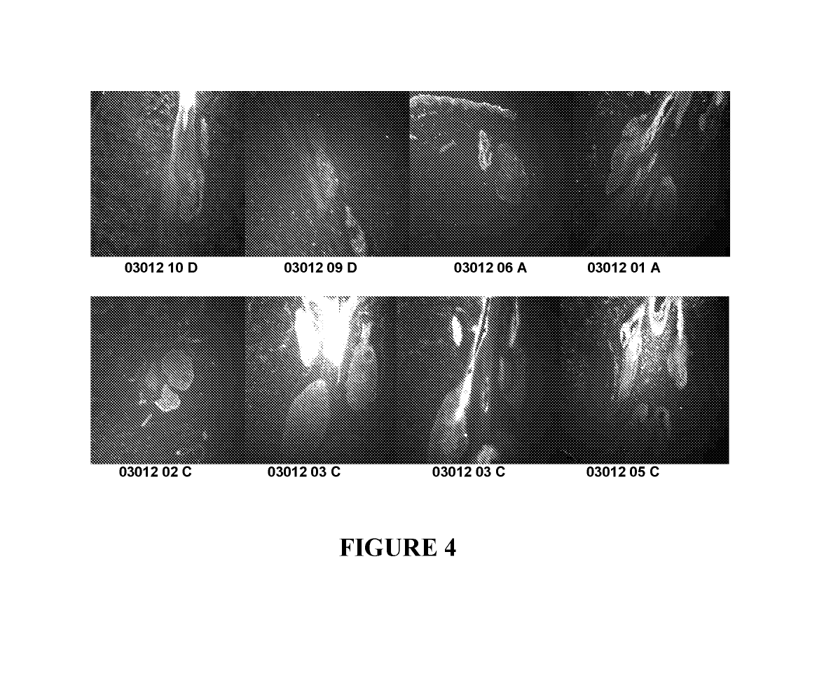

The test group with the highest number of positive biopsies, as defined as a biopsy sample with at least two fluorescence-positive slides sets from all sets evaluated, was Cohort 2 (0.1% LTS). For LTS at 0.1%, 6 of 9 of evaluable (sebaceous gland-containing) biopsies were deemed positive for sebaceous gland fluorescence (see FIG. 4 for fluorescence images of sebaceous glands). For the group that received IR heat treatment plus LTS at 0.1%, 7 of 9 evaluable biopsies were judged to be positive for drug-specific sebaceous gland fluorescence. For subjects treated with LTS at 0.1% in comparison to LTO at 0.2% under occlusion, there was a significant difference in the proportion of group samples exhibiting sebaceous gland lemuteporfin-specific fluorescence as determined by Chi-square statistical analysis (X2 value=15, 3 degrees of freedom, P=0.002). Overall, subjects treated with LTS 0.1%, either alone or with IR heat pre-treatment, exhibited greater extent back skin sebaceous gland fluorescence than when MDA plus LTS at 0.1% or LTO 0.2% under occlusion following IR heat treatment were performed.

These data support the following conclusions. LTS enables distribution of lemuteporfin to the human sebaceous gland, as evidenced by the fact that in subjects administered LTS, lemuteporfin was observed in ≧50-70% of biopsies and 17-45% of biopsy slides via fluorescence microscopy. LTS enables improved distribution of lemuteporfin to the sebaceous gland relative to LTO, as evidenced by the fact that biopsy samples and slides were more frequently positive in subjects administered LTS than LTO under similar conditions (notwithstanding the fact that the concentration of lemuteporfin was 2 to 10-fold lower in LTS than in LTO). Higher concentrations of LTS enable better distribution to the sebaceous gland, as evidenced by the fact that biopsy samples and slides were more frequently positive in subjects administered 0.1% than 0.02% LTS. “Preparing” the skin by administering heat or microdermabrasion prior to applying LTS may not necessarily improve lemuteporfin distribution to the sebaceous gland, as evidenced by the fact that the frequency of positive biopsy samples and slides was not significantly higher in subjects who received such skin preparation procedures than in subjects who did not.

TABLE 11
Fluorescent Image Analysis Results

| | Cohort 1 (LTS 0.02%) | | | | Cohort 1 (LTS 0.1%) | | | |
|---|---|---|---|---|---|---|---|---|
| | | LTS + | LTS + | 0.2% LTO + | | LTS + | LTS + | 0.2% LTO + |
| | LTS | MDA | Heat | Heat | LTS | MDA | Heat | Heat |
| Number of positive | 5 | 4 | 6 | 4 | 6^{a} | 5 | 7^{b} | 3 |
| Biopsies per group of 10 | | | | | | | | |
| Fluorescent positive slides | 17.3 | 18.3 | 24.3 | 19.2 | 41.3 | 22.4 | 44.8 | 19.1 |
| (% slides) | | | | | | | | |

^{a}Excludes 2 negative biopsies, each with 1 slide showing a strong fluorescent signal in sebaceous glands
^{b}Excludes 1 biopsy with no sebaceous gland, and 1 biopsy with only 3 slides with sebaceous gland structure
MDA: Microderabrasion

### Example 9

### Determination of Sebum Excretion Ratio (SER) on the Forehead of a Subject

A sebum excretion ratio may be used to monitor the efficacy of treatment of a subject, and may be determined as follows.

1. Degrease the subject's forehead, by doing the following:
Moisten a cosmetic pad with water.
Apply shampoo to the pad (use an amount about the size of a quarter) and fold the pad in half to distribute the shampoo.
Wash the forehead gently using small circular motions, moving from the middle of the forehead to the temple. Repeat once on each side.
Wipe the forehead gently with water-moistened gauze.
Pat the forehead dry with a clean cosmetic pad.
Wipe the forehead with 70% isopropyl alcohol, working from the center of the forehead to the temple. Use 3 isopropyl alcohol pads for each side of the forehead, wiping the bottom half of the forehead with one pad, the top half with another, then unfold a third pad and wipe the entire side of the forehead.
Let dry at least 5 minutes.

2. Carefully lift the SebuTape™ patch from the carrier sheet and apply to the site, ensuring that the tape is applied smoothly to the skin surface with no wrinkles. Press firmly to ensure the tape is in good contact with the surface of the skin. After 30 min to 120 min (depending on the protocol), remove the patch and transfer the black rectangles on the storage card. Be certain to record the correct date, time and side the patch was applied to (i.e., left or right) in the comment section below the patch.

3. Scan the storage card immediately after sampling with an image resolution of 600 dpi. Save each image file in JPEG format into the appropriate folder using a descriptive filename.

4. Using the appropriate software (e.g., PhotoShop), select all the dark pixels on the patch. Sebum Output is represented by the black pixels which could then be converted to Sebum Excretion Rate by multiplying by a factor of 807.5.

### Example 10

### Stability of Supersaturated Solutions of Lemuteporfin Up to 4 Hours

### A. Stability of LTS Formulation for Vial 1 Solvent Consisting of Benzyl Alcohol

Three formulations (Batches C (Table 8), D (Table 9) and H (Table 10)) in which the Vial 1 photosensitizer component consisted of benzyl alcohol and lemuteporfin at three lemuteporfin concentrations, 0.1, 0.075 and 0.05% w/w in the final combined LTS solution were examined for stability after reconstitution with the remaining excipients in Vial 2.

Vial 2 contents were added to vial 1 for each formulation, mixed and sampled at time 0 and 4 hours after reconstitution. The samples were filtered through a 0.22 μm filter before analysis by HPLC. This analysis was performed to ensure that the combined product had adequate stability and would not precipitate before administration to a subject. The data are presented in Table 12.

TABLE 12
LTS Batches C, D and H Reconstituted Solution

| | Target | Lemuteporfin |
|---|---|---|
| Samples | % w/w | % w/w |
| C - 0.1% w/w | 0.1 | 0.1064 |
| Mixed and filtered | | |
| time 0 | | |
| C - 0.1% w/w | 0.1 | 0.1065 |
| Mixed and filtered | | |
| time 4 hours | | |
| D - 0.075% w/w | 0.075 | 0.0765 |
| Mixed and filtered | | |
| time 0 | | |
| D - 0.075% w/w | 0.075 | 0.0769 |
| Mixed and filtered | | |
| time 4 hours | | |
| H - 0.05% w/w | 0.05 | 0.0503 |
| Mixed and filtered | | |
| time 0 | | |
| H - 0.05% w/w | 0.05 | 0.0504 |
| Mixed and filtered | | |
| time 4 hours | | |

The reconstitution data demonstrated that up to 4 hours post-reconstitution, lemuteporfin is still dissolved and has not precipitated out of the LTS solution for the formulations tested.

### B. Stability of LTS Formulation for Vial 1 Solvent Consisting of DGME and Benzyl Alcohol

Two formulations were examined in which the photosensitizing component in Vial 1 consisted of DGME, benzyl alcohol and lemuteporfin at two lemuteporfin concentrations of 0.1 (Batch A, Table 6) and 0.075% (Batch B, Table 7) in the final formulation. Vial 2 contents were added to Vial 1 contents, mixed and sampled at 0 and 4 hours after reconstitution. The samples were 0.2 μm filtered before analysis. This analysis was performed to ensure that the combined product had adequate stability and would not precipitate before administration to a subject. The data are presented in Table 13.

TABLE 13
LTS Batches A and B Reconstituted Solution

| | Target | Lemuteporfin |
|---|---|---|
| Samples | % w/w | % w/w |
| A - 0.1% w/w | 0.1 | 0.099 |
| Mixed and filtered | | |
| time 0 | | |
| A - 0.1% w/w | 0.1 | 0.100 |
| Mixed and filtered | | |
| time 4 hours | | |
| B - 0.075% w/w | 0.075 | 0.076 |
| Mixed and filtered | | |
| time 0 | | |
| B - 0.075% w/w | 0.075 | 0.077 |
| Mixed and filtered | | |
| time 4 hours | | |

The reconstitution data demonstrate that up to 4 hours, lemuteporfin is still dissolved and has not precipitated out of solution.

We have also found that the chemical stability of the lemuteporfin in Vial 1 of Batch C extends to at least twelve months at 5° C., and at least 6 months at 40° C.

### Example 11

### PDT Treatment of Acne in a Human Subject Using Lemuteporfin PDT

(a) A Lemuteporfin Topical Solution (0.1% LTS) as in Table 8 (Batch C) is applied to an area on the back of a subject having inflammatory acne lesions on the back. The contents of combined Vials 1 and 2 (8.9 g in about 10 ml) are applied to about 300 cm² of skin surface area. Approximately 60 minutes later, the area is exposed to 50 J/cm² of light from an LED light source at 688 nm, delivered at a fluence rate of 50 mW/cm². The sebum excretion rate is determined before therapy, and 2 weeks and 4 weeks thereafter.

(b) Lemuteporfin PDT is conducted as in (a) above, except that the concentration of lemuteporfin in the LTS solution is 0.2%.

(c) Lemuteporfin PDT is conducted as in (a) above, except that the light is administered 15 minutes after LTS application.

(d) lemuteporfin PDT is conducted as in (a) above, except that the area is exposed to 200 J/cm² of light delivered at a fluence rate of 80 mW/cm cm².

Having now fully described the inventive subject matter, it will be appreciated by those skilled in the art that the same can be performed within a wide range of equivalent parameters, concentrations, and conditions without departing from the spirit and scope of the disclosure and without undue experimentation. While the disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

1. A pharmaceutical composition useful for localizing a photosensitizer to a sebaceous gland comprising
(a) a photosensitizing component comprising a photosensitizer; and
(b) an excipient component
in a solution, wherein the concentration of said photosensitizer in the solution is supersaturating.

2. The composition of claim 1, wherein said photosensitizer is present at a concentration in the range of about 0.01% to about 1.0%.

3. The composition of claim 2, wherein said photosensitizer is present at a concentration in the range of about 0.025% to about 0.5%.

4. The composition of claim 3, wherein said photosensitizer is present at a concentration in the range of about 0.1% to about 0.2%.

5. The composition of claim 1, wherein said excipient component comprises benzyl alcohol at a concentration in the range of about 1% to about 20%.

6. The composition of claim 5, wherein said benzyl alcohol concentration is about 10%.

7. The composition of claim 1, wherein said excipient component comprises diethylene glycol monoethyl ether (DGME) at a concentration in the range of about 5% to about 50%.

8. The composition of claim 7, wherein said excipient DGME concentration is in the range of about 15% to about 35%.

9. The composition of claim 1, wherein said excipient component comprises isopropyl alcohol at a concentration in the range of 40 to 70%.

10. The composition of claim 1, wherein said photosensitizer is a green porphyrin.

11. The composition of claim 10, wherein said green poprhyrin is lemuteporfin.

12. A topical formulation effective for localizing a photosensitizer to a sebaceous gland comprising:
(a) a photosensitizing component comprising a photosensitizer; and associated therewith but separate therefrom,
(b) an excipient component;
wherein said photosensitizer is present in an amount sufficient to form, on mixing, a supersaturated solution thereof once components (a) and (b) are mixed.

13. A topical formulation of claim 1 for use in a method of treating acne in a subject in need thereof, comprising applying a therapeutically effective amount of said composition to an affected area of a subject's skin having acne lesions, allowing sufficient time for at least some of said photosensitizer localize in the sebaceous glands of said affected area, and exposing said skin of said subject to light energy at a wavelength capable of activating said photosensitizer.

14. A method comprising the steps of:
(a) providing a photosensitizing component comprising a photosensitizer dissolved in a solvent;
(b) providing an excipient component miscible with said photosensitizing component; and
(c) mixing an amount of said photosensitizing component with an amount of said excipient component to provide a mixed solution,
wherein said mixed solution is supersaturated with said photosensitizer.

15. The method of claim 14, wherein said photosensitizing component comprises benzyl alcohol, and said photosensitizer is a green porphyrin.

16. The method of claim 15, wherein said green porphyrin is selected from lemuteporfin or verteporfin.

17. A photosensitizer composition of claim 1 for use in a method for reducing the sebum excretion rate of sebaceous glands in the skin of a subject having an affected area of oily skin, comprising applying a therapeutically effective amount of said photosensitizer composition to said affected area, allowing sufficient time for at least some of said photozensitizer composition to localize in said sebaceous glands, and exposing said skin of the subject to light energy at a wavelength capable of activating said photosensitizer.
